(19) United States
(12) Patent Application Publication
(10) Pub. No.: US 2021/0330411 A1
(43) Pub. Date: Oct. 28, 2021

(54) PROTECTIVE ENVELOPE FOR A ROBOTIC MODULE FOR DRIVING A FLEXIBLE ELONGATE MEDICAL ELEMENT WITH GUIDE TRACK

(71) Applicant: ROBOCATH, ROUEN (FR)

(72) Inventors: Fabien DESTREBECQ, BOURGTHEROULDE (FR); Bruno FOURNIER, SAINT OUEN (FR); Philippe BENCTEUX, ST MARTIN DU VIVIER (FR)

(21) Appl. No.: 16/613,897

(22) PCT Filed: Mar. 29, 2018
(86) PCT No.: PCT/FR2018/050790

§ 371 Date: Nov. 15, 2019

(30) Foreign Application Priority Data
May 16, 2017 (FR) 1754306

(51) Int. Cl. A61B 46/10; A61B 34/30; A61B 50/00; A61M 25/01; A61M 25/09

(52) U.S. Cl. CPC A61B 46/10; A61B 34/30; A61B 50/00; A61B 2050/005; A61M 25/09041; A61B 2034/301; A61M 25/0113

(57) ABSTRACT

The protective envelope for a robotic module for driving a flexible elongate medical element, intended to form a barrier between the robotic driving module and the flexible elongate medical element, includes: —a mechanism for attachment to the robotic module, and —a guide track for the flexible elongate medical element including a longitudinal axis and a main portion having a free surface of concave shape, the main portion including a longitudinal groove for receiving the flexible elongate medical element, extending along the longitudinal axis and hollowed out in the free surface having a concave shape.

### FIELD OF THE INVENTION

The invention relates to robotic modules for driving flexible elongate medical elements.

More particularly, the invention relates to the insertion of a guide wire, of a catheter guide, and of the catheter itself, in particular for the treatment of cardiovascular diseases, into a patient's artery. A catheter guide is a flexible elongate medical element of small diameter, generally non-hollow, onto which a catheter is threaded. A catheter is a flexible elongate medical element of larger diameter, generally hollow, that can be threaded onto the guide. The catheter may provide a medical function, generally at its end inserted into the patient, such as a tool or a particular shape.

In practice, the invention applies to any medical member that is sufficiently flexible to be movable within a physiological passageway of a human patient, and sufficiently elongate so that its end can be moved about from outside the patient.

### TECHNOLOGICAL BACKGROUND OF THE INVENTION

The manual insertion of a catheter into a patient is a relatively conventional surgical procedure. However, as this procedure is performed using X-rays, the surgeon in charge of the procedure is exposed to significant radiation if he or she performs such an operation on numerous patients.

In order to reduce the risks for the surgeon, it has been attempted to robotize such insertion by using a robotic module to drive the catheter. However, such robotization is complex because it is difficult to grip the catheter: the catheter is bathed in preservation liquid and must remain sterile. Moreover, the ability to control alternating and/or simultaneous movements of the catheter in translation and in rotation is desired. In addition, smaller catheters may have a diameter of less than 0.25 mm. The reliability of the driving module is therefore a determining factor.

The reliability of the driving module is based, among other factors, on the fact that the catheter is in a sterile state when inserted into the patient. However, the robotic driving module, which is expensive, is intended to be used to operate on a large number of patients and to be stored between uses. The robotic driving module may therefore come into contact with microorganisms or dust.

This is why, in order to avoid soiling the catheter and/or the robotic module, a sterile, disposable protective envelope is placed on the robotic driving module before each procedure. Document WO 2015/189529 describes such a protective envelope. The envelope at least partially covers the robotic driving module and contributes to forming a sterile barrier between the driving module and the catheter. Movement is transmitted from the robotic module to the catheter, or to the catheter guide, through the envelope.

Only the protective envelope is in direct contact with the catheter and guide during the procedure of insertion into the patient. Therefore, it usually carries a guide track for the guide or catheter.

The guide track for the catheter provides a surface to carry for example the catheter or catheter guide and enabling the latter to be moved along a rectilinear track which thus defines a longitudinal axis. This track forms a tube having a diameter greater than that of the flexible elongate medical element. The tube is formed by two half-tubes that can be detached from one another, their separation surface located in a substantially horizontal plane passing through the axis of the tube, enabling the placement or removal of the catheter or guide into or from the guide track.

When the tube is open, the surgeon must be able to place the catheter or guide correctly inside, even though the diameter of the tube is smaller than a surgeon's finger. When the tube is closed, the catheter or guide must be properly held in the tube. Indeed, if the diameter of the tube is too large, the catheter or guide may not remain straight while moving. If the tube is too small in diameter, the catheter or guide will rub excessively against its walls which could cause damage.

Contradictory constraints are thus imposed on the tube that forms the guide track.

### OBJECT OF THE INVENTION

An object of the invention is therefore to provide a more reliable protective envelope that allows precise positioning of the flexible elongate medical element which may in particular be a catheter or a guide.

### BRIEF DESCRIPTION OF THE INVENTION

To achieve this, the invention provides a protective envelope for a robotic module for driving a flexible elongate medical element, intended to form a barrier between the robotic driving module and the flexible elongate medical element, characterized in that it comprises:

means of attachment to the robotic module, and

a guide track for the flexible elongate medical element, comprising a longitudinal axis and a main portion having a free surface of concave shape, the main portion comprising a longitudinal groove for receiving the flexible elongate medical element, extending along the longitudinal axis and hollowed out of the free surface having the concave shape.

The concave shape of the free surface of the main portion thus allows a user, in particular a surgeon, to manually position the flexible elongate medical element in the longitudinal groove before beginning the operation of insertion into the patient's body. The groove receives the flexible elongate medical element and the concave shape allows the user to lock the flexible medical element elongate in the groove by the simple pressure of a finger. The placement of the flexible elongate medical element in the envelope is therefore simpler and more reliable.

Preferably, the longitudinal groove has a free surface for which the cross-section has a “V” or “U” shape.

Such a shape is adapted to receive the flexible elongate medical element. We will clarify here that it is the groove, which has dimensions suitable for positioning the flexible elongate medical element, which has a free surface whose cross-section has a “V” or “U” shape, and not the free surface of the guide track as a whole.

Advantageously, the longitudinal groove is part of a plane of symmetry of the free surface having a concave shape.

The envelope is therefore simple to manufacture, in particular by injection molding.

According to one embodiment, the free surface having a concave shape has a radius of curvature comprised between 0.5 and 3 cm, or a radius of curvature comprised between 1 and 2 cm.

Such a range for the radius of curvature generally follows the shape of the lower surface of the tip of an index finger. Manual placement of the catheter in the longitudinal groove is therefore all the more simple.

Preferably, the main portion comprises at least one transverse groove extending perpendicularly to the longitudinal axis, hollowed out in the free surface having a concave shape.

The transverse groove is able to receive complementary positioning means of the flexible elongate medical element, which improves the reliability of the positioning of the flexible elongate medical element.

Advantageously, the guide track comprises a second portion which comprises at least one support member for the flexible elongate medical element, of which a free surface comprises two arms which each have a convex shape and which define a slot adapted to receive the flexible elongate medical element.

Thus, by installing the flexible elongate medical element on free surfaces of concave and convex shapes, the member is positioned even more reliably.

Preferably, the free surface of the support member for the flexible elongate medical element is offset, in a direction perpendicular to the longitudinal axis of the guide track, relative to the free surface having a concave shape.

According to one embodiment, the envelope comprises at least two transverse grooves, the support member for the flexible elongate medical element being arranged, along the longitudinal axis, between the two transverse grooves.

The catheter is thus made more taut for the same overall length of the guide track. The reliability of the catheter positioning is therefore improved.

Advantageously, the envelope comprises a removable cover able to occupy an open position where the cover allows access to the guide track and a closed position where the cover at least partially encloses the guide track.

The cover thus protects the guide track and the catheter when it is present.

Preferably, the cover and the guide track comprise complementary guide members for the flexible elongate medical element.

Thus, when the cover is in the closed position, the complementary guide members for the flexible elongate medical element cooperate to grip the catheter on all sides and thus position it more precisely.

Advantageously, the guide member carried by the cover has a free surface which has a “W”-shaped cross-section.

This free surface comes to surround the flexible elongate medical element in order to position it with even more precision.

Preferably, the guide member is configured to be positioned in the transverse groove when the cover is in the closed position.

According to one embodiment, the envelope comprises electrically-controlled means for locking the cover in the closed position.

Preferably, the means for locking the cover in the closed position are connected to a presence sensor able to lock the cover in the closed position when the presence sensor detects a presence close to the protective envelope.

This improves the reliability of the positioning of the flexible elongate medical element.

Advantageously, the envelope comprises a pair of drive members for the flexible elongate medical element, the drive members being paired opposite one another, one on either side of the longitudinal axis of the guide track.

Also provided according to the invention is an assembly comprising a robotic module for driving a flexible elongate medical element and a protective envelope, as described above, the envelope being fixed to the robotic driving module so as to form a barrier separating a space comprising the robotic module from a space comprising the flexible elongate medical element.

Advantageously, the protective envelope is removably attached to the robotic module.

Preferably, the robotic driving module is configured to drive a catheter guide and/or a catheter through the envelope.

### BRIEF DESCRIPTION OF THE DRAWINGS

We will now describe one embodiment of the invention as a non-limiting example, with the aid of the following figures:

FIG. 1A is a schematic side view of a robotic arteriography system,

FIG. 1B is a top view of a portion of FIG. 1A,

FIGS. 2A to 2C are diagrams illustrating the modes of movement of the flexible elongate medical elements,

FIG. 3 is a top view of a protective envelope for a robotic module of the arteriography system,

FIG. 4 is a top perspective view of the protective envelope,

FIG. 5 is a front view of the protective envelope,

FIGS. 6 and 7 are respective front and back views of a drive member for the protective envelope,

FIG. 8 is a top perspective view of a protective envelope comprising a cover,

FIGS. 9A to 9C illustrate cross-sections of portions of the protective envelope and cover,

FIGS. 10A and 100 show support members and complementary guiding members during closure of the cover of the protective envelope, and

FIGS. 11A to 11F illustrate cross-sections of the complementary guiding members of the cover of the protective envelope according to a variant of the embodiment described, during a progressive closure of the cover.

### DETAILED DESCRIPTION OF AN EMBODIMENT OF THE INVENTION

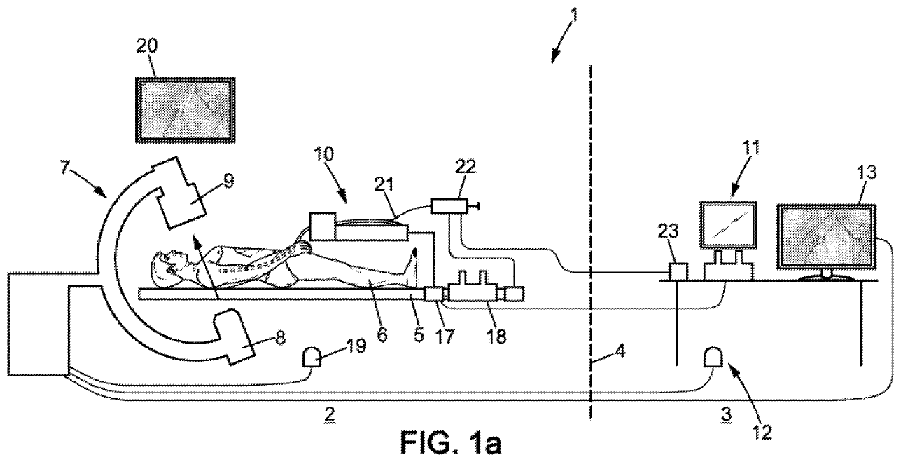

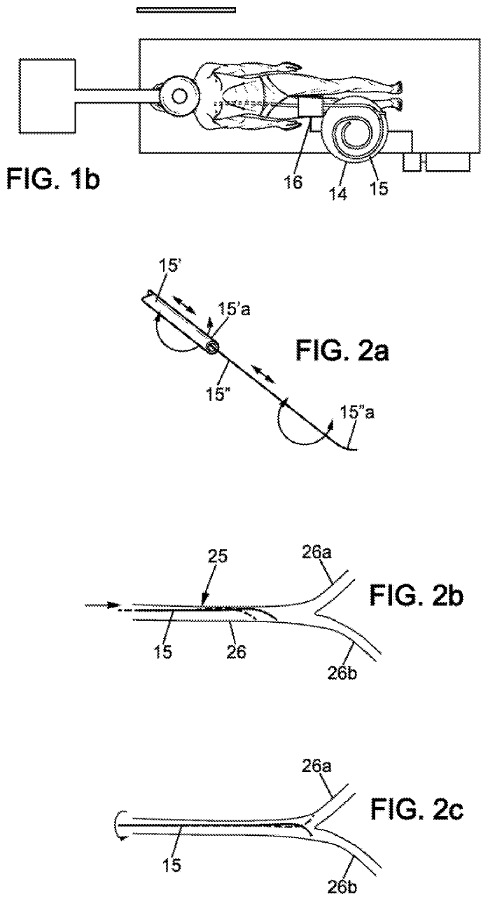

FIG. 1a schematically represents an arteriography system 1. The arteriography system 1 is divided into two separate locations, an operating room 2 and a control room 3. The control room 3 may be close to the operating room 2, separated from it by a simple wall 4 that is opaque to X-rays, or remote. The equipment of the operating room 2 and control room 3 are functionally interconnected by wires, wirelessly, and/or by network. The control room can thus, and preferably, be a simple area inside the operating room, defined by a radiation shielding screen providing the physician with an area protected from X-rays.

The operating room 2 comprises an operating table 5 receiving a patient 6. The operating room 2 may also comprise a medical imager 7, in particular an X-ray imager, comprising a source 8 and a detector 9 arranged one on each side of the patient 6, possibly movable relative to the patient 6.

The arteriography facility 1 comprises a robot 10 arranged in the operating room 2.

The arteriography facility 1 comprises a control station 11 arranged in the control room 3. The control station 11 is suitable for controlling the robot 10 remotely.

The arteriography facility 1 may also comprise, arranged in the control room 3, one or more remote controls 12 for the imager 7, communicating with the imager 7 in order to control it remotely. The arteriography facility 1 may also comprise a screen 13, arranged in the control room 3, communicating with the imager 7, for real-time viewing in the control room 3 of the images acquired by the imager 7.

The robot 10 may comprise a container 14 adapted to contain a flexible elongate medical element 15 to be inserted into the body of a patient. This container 14 may in particular be arranged inside the robot. For a flexible elongate medical element 15, it may for example be a member to be inserted into a patient passageway and to be moved within this passageway, in particular a patient's artery or vein, through an introducer providing an access opening in the patient. The flexible elongate medical element 15 may in particular be a catheter or a guide. A guide generally has a smaller transverse diameter than that of the catheter, which is generally hollow in a portion close to the patient or even for its entire length, so that the guide can move inside it, in particular inside the patient's body.

The robot 10 comprises a robotic driving module 16 for the flexible elongate medical element 15. The robotic driving module 16 can be controlled from the control station 11 to drive the flexible elongate medical element 15 relative to the patient 6 according to two degrees of freedom, as will be described in detail below. The robotic driving module 16 comprises a communication unit 17 for interfacing with the control station 11. If necessary, the robot 10 may comprise a local control unit 18, for controlling the robot from the operating room 2 if necessary.

One will note that all the commands and feedback available in the control room 3 may also be available in the operating room 2 for a local operation, for example such as a control 19 for the imager and a screen 20 for displaying images acquired by the imager 7.

In the following, the reference 15 will be used to designate the flexible elongate medical element which may be an interventional catheter or a guide wire. Such an interventional catheter may have a smaller diameter than the guide catheter, so that it can coaxially guided therein, inside the patient, and may be hollow so that it can be guided on the guide inside the patient.

Optionally, the hollow flexible elongate medical element may be connected to a connector 21 to allow injecting a contrast medium facilitating imaging inside the flexible elongate medical element. The arteriography facility may comprise a contrast medium injector 22 connected to the connector 21, controllable by a control 23 provided in the control room 3.

FIG. 2A shows the various degrees of freedom possible with the present system. The guide 15″ can be seen with its front end slightly curved relative to the main longitudinal axis of the guide, and exiting at the front end of the catheter 15. The catheter 15′ can be subjected to two distinct movements: a translation along its longitudinal axis and a rotation about its longitudinal axis. These movements can be generated in one direction or in the other. If appropriate, the catheter 15′ can be subjected to a combined movement of the two simple movements described above.

Where appropriate, the catheter 15′ may be subjected to two combined movements of the two simple movements described above, in different combinations.

What has been described above regarding the catheter also applies to the guide.

In FIG. 2B an artery 25 of a patient is represented, comprising a main trunk 26 and two branches 26A, 26B leading off the main trunk. FIG. 2B illustrates the movement of a flexible elongate medical element 15 (here a guide 15″) in translation between a retracted position shown in dashed lines and an advanced position shown in solid lines. In FIG. 2C, in the same artery, a rotation of the flexible elongate medical element 15 is represented, between a first position shown in dotted lines, where the flexible elongate medical element is ready to be moved in translation in the direction of branch 26A, and a second position shown in solid lines, where the flexible elongate medical element is ready to be moved in translation in the direction of branch 26B. The catheter 15′ and the catheter guide 15″ are set in motion by means which will be described below.

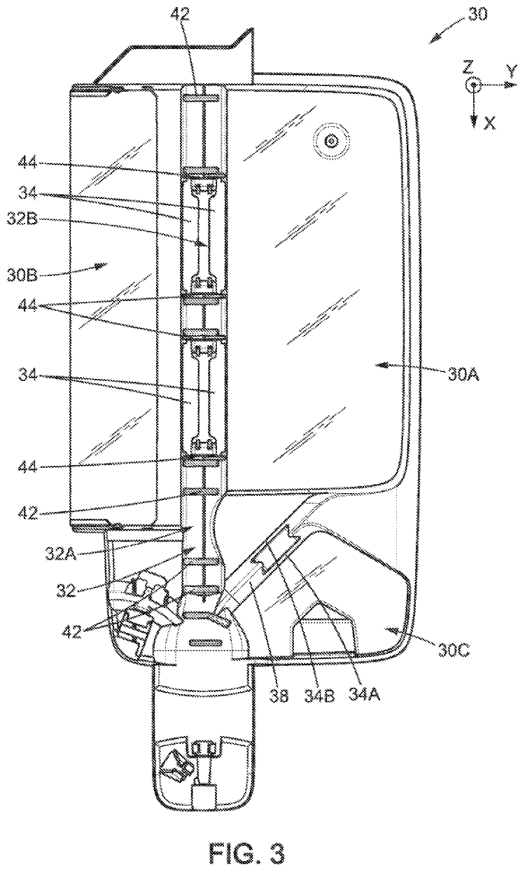

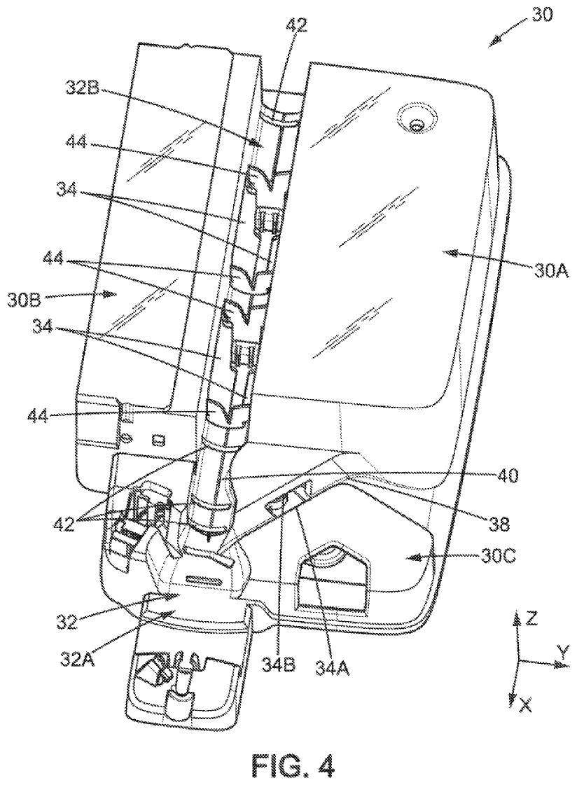

FIGS. 3 to 5 and 8 show a protective envelope 30 according to the invention. The protective envelope 30 is intended to form a barrier between the robotic driving module 16 and the flexible elongate medical element 15. For this purpose, the robotic driving module 16 comprises a articulated arm, not shown, which is covered by the protective envelope 30. It is possible that another member is interposed between the protective envelope 30 and the articulated arm.

The robotic driving module 16 is specifically configured to drive a guide wire, a guide catheter, or a catheter, as previously indicated, through the protective envelope 30. To do this, the protective envelope 30 may be removably attached to the robotic driving module 16. More generally, the protective envelope comprises means of attachment to the robotic driving module 16 which may be of any type. These means may for example comprise clips, tenons, catch pins. The protective envelope 30 thus forms a sterility barrier which separates a space comprising the robotic driving module 16 from a space which comprises the flexible elongate medical element 15 and which is therefore a manipulation space for the flexible elongate medical element 15.

The protective envelope 30 has an upper surface, along a vertical direction (Z), when the protective envelope 30 is positioned on the robotic driving module 16, which is visible in FIG. 3 in particular. It is this upper surface which carries the flexible elongate medical element 15 as will be described below. A lower surface, in the opposite vertical direction (Z), is in contact with the robotic driving module 16.

The protective envelope 30 has two parts 30A and 30B which extend in the three non-coplanar dimensions of space, and which are arranged one on each side of a guide track 32 for the flexible elongate medical element 30. Portions 30A and 30B of the protective envelope 30 comprise thin horizontal and vertical walls substantially forming a “semi-parallelepiped” such that the protective envelope 30 forms a protective shell.

Each portion 30A, 30B comprises a main cavity accessible by two windows 34 carried by a wall which extends vertically. Each window 34 carried by one cavity is opposite a window carried by the other cavity. In addition, the two portions 30A and 30B form a trough between the four windows 34, said trough forming a portion of the guide track 32.

The first four windows 34 of the main body 32 have dimensions adapted to receive drive members 36, visible in FIGS. 6 and 7 and described in more detail below. These are arranged so that they are paired opposite one another, one on each side of the guide track 32.

In addition, the protective envelope 30 comprises a second guide track 38, formed between portion 30A and a third portion 30C of the protective envelope 30 which also extends in the three non-coplanar dimensions of space. The guide track 38 is provided to guide a second flexible elongate medical element, for example a catheter. Thus, the protective envelope 30 makes it possible to simultaneously introduce two flexible elongate medical elements 15, for example the catheter and the guide. The third portion 30C further comprises a fifth window 34A for receiving a drive member, not shown here, for the flexible elongate medical element 15 positioned on the second guide track 38. The drive member may in particular be a roller. In this case, a sixth window 34B in portion 30A, intended to accept a counter-roller, is arranged facing the fifth window 34A.

The guide track 32 is rectilinear and has the function of ensuring that the flexible elongate medical element 15 remains rectilinear during its movement along a longitudinal direction (X). Indeed, in the absence of this guide track 32, the flexible elongate medical element 15 could form a loop between a pushing zone and the patient entry point. Note also that the longitudinal direction (X) of movement of the flexible elongate medical element 15 is a longitudinal direction of the guide track 32, but is not necessarily a longitudinal direction of the protective envelope 30. In FIGS. 3 to 5 and 8, the longitudinal direction (X) is oriented towards the “forward” direction of movement of the flexible elongate medical element 15, in other words the direction allowing insertion of the flexible elongate medical element 15 into the patient's body.

The guide track 32 comprises a main portion 32A which is an upper end portion, in the longitudinal direction, and a second portion 32B. The main portion 32A and the second portion 32B are arranged one after the other along the longitudinal direction (X). The direction extending from the second portion 32B to the main portion 32A is parallel to the longitudinal direction (X). The second portion 32B is arranged between the two portions 30A and 30B and more specifically between the four windows 34. The main portion 32A is partially arranged between the two portions 30A and 30B and extends beyond.

The main portion 32A has a free surface having a concave or “U” shape. FIG. 9A illustrates this free surface of the main portion 32A. In addition, the main portion 32A comprises a longitudinal groove 40 hollowed out in the free surface of this main portion 32A. The longitudinal groove 40 extends along the longitudinal axis (X). This longitudinal groove 40 has a free surface having a “V”-shaped cross-section and is part of a plane of symmetry (XZ), comprising the longitudinal and vertical directions, of the free surface having a concave shape.

Note that according to a preferred variant of this embodiment, the longitudinal groove 40 has the shape of a “U”, “C”, or circular arc.

The longitudinal groove 40 is intended to receive the flexible elongate medical element 15. Thus, the “V” shape of its free surface forms a housing particularly suitable for receiving the flexible elongate medical element 15. In addition, the “U” or concave shape of the free surface of the main portion 32A of the guide track 32 forms a support for an index or middle finger of a user who can easily press the flexible elongate medical element 15 into the “V”-shaped groove. For this purpose, the radius of curvature of the free surface of concave shape is comprised between 0.5 cm and 1.5 cm.

In addition, this main portion 32A of the guide track 32 comprises a plurality of transverse grooves 42 which extend in a transverse direction (Y) perpendicular to the longitudinal (X) and vertical (Z) directions. These transverse grooves 42 are formed in the free surface as a concave or “U” shape and form slots for receiving other positioning means of the flexible elongate medical element 15 carried by a cover, as will be described below.

The second portion 32B of the guide track 32 comprises a plurality of support members 44, here four in number. These four support members 44 are spaced apart and arranged one after the other along the longitudinal direction (X). In addition, along this longitudinal direction (X), two respective support members 44 flank two windows 34 that face one another. These support members 44 have a free surface, visible in particular in FIG. 9B, which has two arms 44A and 44B extending in the transverse direction (Y) and symmetrically arranged relative to the longitudinal direction (X) of the guide track 32. Each arm 44A, 44B has a convex shape. The meeting point of the two arms 44A, 44B thus forms a slot serving to receive the flexible elongate medical element 15.

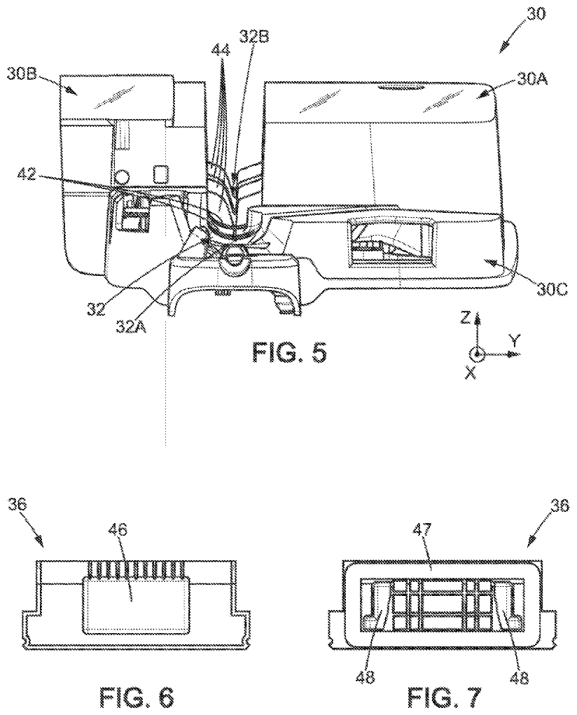

In addition, as can be seen in particular in FIG. 5, in the vertical direction (Z), the free surfaces of the support members 44 are offset relative to each other. The support member 44 which is closest to the main portion 32A of the guide track 32 has a free surface where the slot is at the same level as the free surface of this main portion 32A, having a “U” or concave shape. Conversely, the support member 44 which is farthest from the main portion 32A has a free surface where the slot is raised relative to the free surface of this main portion 32A. In addition, at its lower end in the longitudinal direction (X), the second portion 32B comprises a transverse groove 42. Thus, along the longitudinal direction (X), the support members 44 are arranged between two transverse grooves 42.

Thus, the “V”-shaped longitudinal groove 40 and the support members 44 form positioning means for the flexible elongate medical element 15 which still allow said member to be movable in translation and in rotation. Indeed, it is movable in translation along the longitudinal direction (X) from front to back and vice versa, or in rotation about the longitudinal direction (X) in one direction or the other, by means of the drive members 36 illustrated in FIGS. 7A and 7B.

Each drive member 36 has a generally parallelepipedal shape and comprises a drive pad 46. Thus, each pair of drive members 36 is able to clamp the flexible elongate medical element 15 between two drive pads 46 in order to set it into motion. On a surface opposite the surface which carries the drive pad 46, there is provided a bead of adhesive 47 located on a peripheral perimeter of this surface. Thus, the bead of adhesive 47 provides a fluidtight attachment of the drive members 36 to the respective perimeters of the windows 34. According to one variant, the bead of adhesive is associated with or replaced by double-sided adhesive tape. Thus, the drive members 36 pass through the windows 34 of portions 32A and 32B and are sealingly positioned with respect to the guide track 32. In addition, each drive member 36 is connected, by means of two anchoring tabs 48, to an outlet end of the robotic driving module 16 which can be controlled by an actuator to move rapidly, cyclically, and locally, within the space. The outlets of the robotic driving module 16 and the actuators that control them are carried by the articulated arm, which allows a movement of greater magnitude relative to the general dimensions of the articulated arm of the robotic driving module 16.

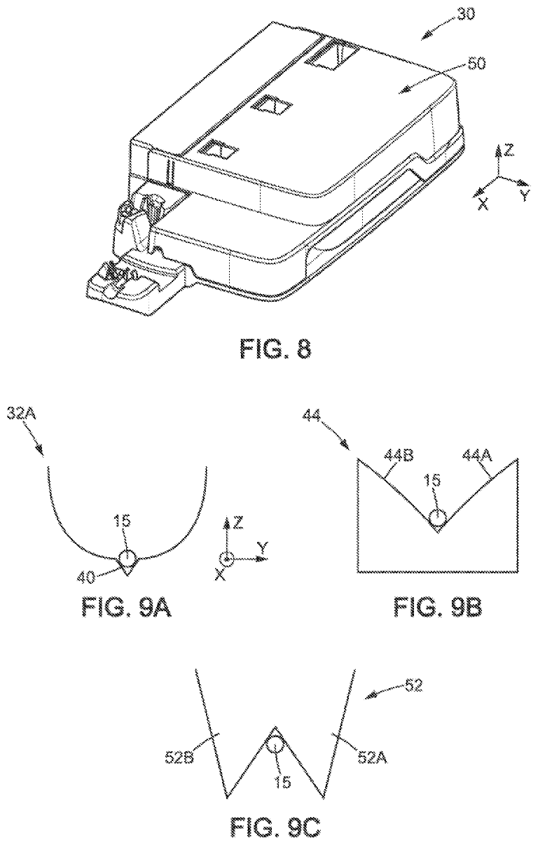

In addition, optionally and preferably, the protective envelope 30 comprises a removable cover 50 adapted to occupy an open position where it allows access to the guide tracks 32 and 38, and a closed position illustrated in FIG. 8, where the cover 50 encloses the guide tracks 32 and 38. This cover 50 comprises means for locking in the closed position which can be electrically controlled. In addition, these locking means can be connected to a presence sensor allowing, for example, locking the cover 50 in the closed position when a presence is detected near the protective envelope 30.

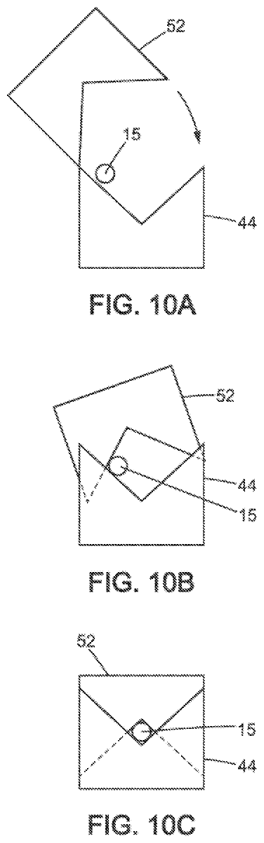

When in the closed position, the cover 50 comprises an upper surface that can be seen in FIG. 8. It also comprises an opposite surface which faces the guide tracks 32 and 38. This opposite surface comprises complementary guide members for the flexible elongate medical element 15. For example, one of the complementary guide members 52, visible in FIG. 9C, comprises a downward-facing free surface 54 relative to the vertical direction (Z) and which has a “W”-shaped or “forked tongue” cross-section. This complementary guide member 52 thus comprises two teeth 52A and 52B, symmetrically arranged relative to the longitudinal direction (X) and configured to be arranged in one of the transverse grooves 42 and to cooperate with the support members 44 when the cover 50 is in the closed position as illustrated in particular in FIGS. 10A to 100. Indeed, as shown in these figures, when the complementary guide members 52 cooperate with the transverse grooves 42 and support members 44, they sandwich the flexible elongate medical element 15 to position it.

More generally, the cover 50 may comprise other complementary guide members which have complementary shapes to the “V”-shaped longitudinal groove 40, transverse grooves 42, or support members 44.

For example, the complementary guide members 52 and the support members 44 may have shapes specifically configured so that no interference remains when the cover 50 is closed and so that the flexible elongate medical element 15 is always correctly positioned in the longitudinal groove 40 regardless of the position of the flexible elongate medical element 15 and the angular position of the envelope 30 during closure of the cover 50.

In this manner, the flexible elongate medical element 15 is positioned to be movable in translation and in rotation, both by the positioning means of the guide track 32 and by positioning means carried by the cover 50.

Of course, many modifications can be made to the invention without departing from the scope thereof.

Any type of positioning member for the flexible elongate medical element 15 may be used.

In particular, the complementary guide members 52 may be connected by a track which travels through the cover 50. The track comprises a cross-section having a “U”, “0”, or circular arc shape, so as to have a shape complementary to the longitudinal groove 40. The envelope 30 thus comprises a substantially cylindrical guide channel.

It is possible for the main portion 32A and the second portion 32B of the guide track 32 to comprise either the transverse grooves 42 or the support members 44.

As many guide tracks 32 as necessary may be provided on the protective envelope 30.

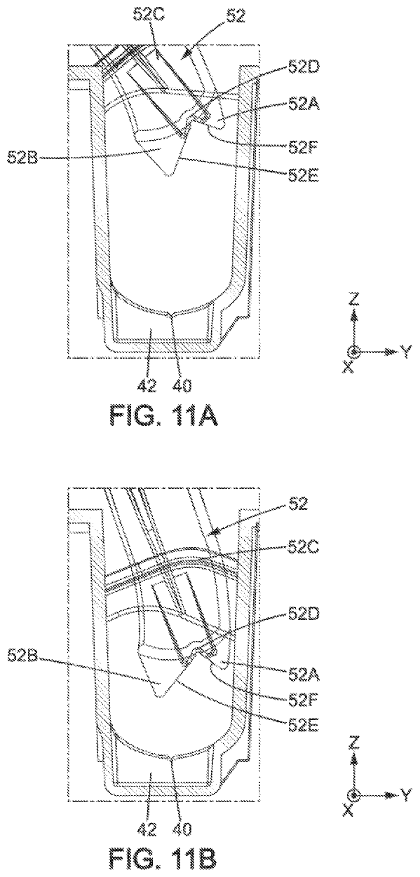

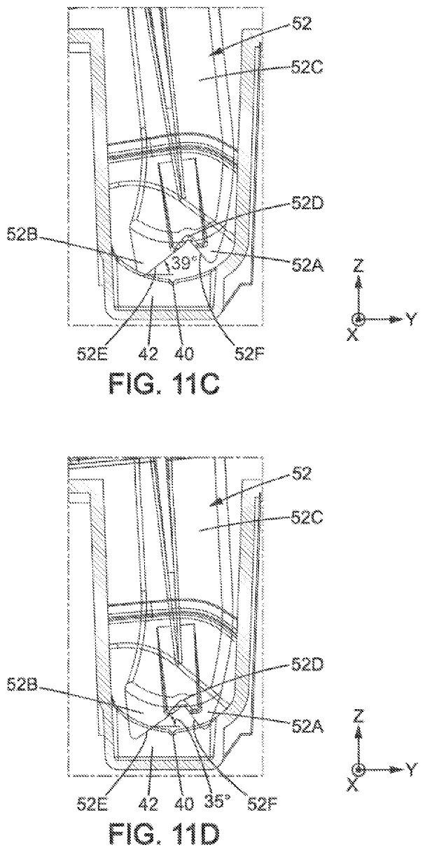

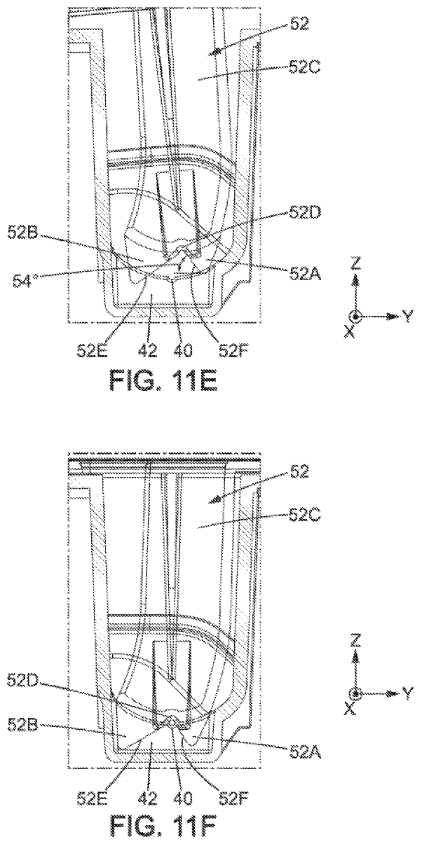

In addition, represented in FIGS. 11A to 110 is a complementary guide member 52 according to an advantageous variant of the present embodiment. The numerical references for similar elements remain unchanged.

The complementary guide member 52 is intended to cooperate with one of the transverse grooves 42 to position the flexible elongate medical element 15 in the longitudinal groove 40.

To do this, the complementary guide member 52 comprises a main body 52C which carries, at its lower end in the vertical direction Z when the cover 50 is in the closed position, two teeth 52A and 52B arranged one beside the other in the transverse direction (Y). However, unlike the complementary guide member 52 described above, the two teeth 52A and 52B are not symmetrically arranged with respect to the longitudinal direction (X). Indeed, tooth 52A, which is arranged on the right in FIGS. 11A to 110 and which is the right tooth when viewing the unit 30 from the patient's head 6, has smaller dimensions than tooth 52B which is the left tooth.

Thus, the two teeth 52A and 52B give the complementary guide member 52 a general asymmetrical “W” shape in plane (ZY).

Specifically, the cross-section of the teeth 52A and 52B in plane (ZY) has a substantially triangular shape. Tooth 52A thus has a substantially triangular shape with its three sides that define the triangular shape having a length less than the three sides that define the triangular shape of tooth 52B. In other words, in plane ZY, the triangular shape of tooth 52A has a smaller area than the area of the triangular shape of tooth 52B.

Furthermore, in plane (ZY), the meeting point between the teeth 52A and 52B comprises a recess 52D having a shape such that, as illustrated in particular in FIG. 11F, the recess 52D is arranged symmetrically relative to the longitudinal groove 40 in the transverse direction (Y) when the cover 50 is in the closed position.

We will now describe the dynamics when closing the cover 50.

As illustrated in FIG. 11A, the cover 50 is closed by swinging it clockwise about a direction parallel to the longitudinal direction (X) when viewing the unit 30 from the patient's head 6.

Thus, as shown in FIGS. 11B and 110, since tooth 52A has smaller dimensions than those of tooth 52B, tooth 52A does not abut against the walls of the unit 30 which surround the longitudinal groove 40. The complementary guide member 52 is thus not impeded in its movement when closing the cover 50 of the unit 30.

This is why the complementary guide member 52 can move relative to the transverse groove 42 according to a dynamic illustrated in FIGS. 11A to 11F. As the cover 50 is closed, the complementary guide member 52 guides the flexible elongate medical element 15 towards the longitudinal groove 40. Indeed, as shown in FIG. 11F, the longitudinal groove 40 and the recess 52D together define a space for receiving the flexible elongate medical element 15.

In addition, in the configuration shown in FIGS. 11D to 11F, tooth 52B has a free surface of which a rectilinear portion 52E, arranged facing tooth 52A, forms an angle with the transverse direction (Y) that is substantially between 35° and 40°. Similarly, tooth 52A has a free surface of which a rectilinear portion 52F, arranged facing tooth 52B, forms an angle with the transverse direction (Y) that is substantially between 50° and 55°. These angles have been chosen so as to effectively guide the flexible elongate medical element 15 towards the space defined by the longitudinal groove 40 and the recess 52D as the cover 50 is closed.

1. Protective envelope (30) for a robotic module (16) for driving a flexible elongate medical element (15), intended to form a barrier between the robotic driving module (16) and the flexible elongate medical element (15), characterized in that it comprises:
means of attachment to the robotic module, and
a guide track (32) for the flexible elongate medical element (15), comprising a longitudinal axis (X) and a main portion (32A) having a free surface of concave shape, the main portion (32A) comprising a longitudinal groove (40) for receiving the flexible elongate medical element (15), extending along the longitudinal axis (X) and hollowed out in the free surface having a concave shape.

2. Envelope (30) according to the claim 1, wherein the longitudinal groove (40) has a free surface for which the cross-section has a “V” or “U” or “C” or circular arc shape.

3. Envelope (30) according to claim 1, wherein the longitudinal groove (40) is part of a plane of symmetry (XZ) of the free surface having a concave shape.

4. Envelope (30) according to claim 1, wherein the free surface having a concave shape has a radius of curvature of between 0.5 and 2 cm.

5. Envelope (30) according to claim 1, wherein the main portion (32A) comprises at least one transverse groove (42) extending perpendicularly to the longitudinal axis (X), hollowed out in the free surface having a concave shape.

6. Envelope (30) according to claim 1, wherein the guide track (32) comprises a second portion (32B) which comprises at least one support member (44) for the flexible elongate medical element (15), of which a free surface comprises two arms (44A, 44B) which each have a convex shape and which define a slot adapted to receive the flexible elongate medical element (15).

7. Envelope (30) according to claim 6, wherein the free surface of the support member (44) for the flexible elongate medical element (15) is offset, in a direction (Z) perpendicular to the longitudinal axis (X) of the guide track (32), relative to the free surface having a concave shape.

8. Envelope (30) according to claim 6, comprising at least two transverse grooves (42), the support member (44) for the flexible elongate medical element (15) being arranged, along the longitudinal axis (X), between the two transverse grooves (42).

9. Envelope (30) according to claim 1, comprising a removable cover (50) able to occupy an open position where the cover (50) allows access to the guide track (32) and a closed position where the cover (50) at least partially encloses the guide track (32).

10. Envelope (30) according to claim 9, wherein the cover (50) and the guide track (32) comprise complementary guide members (42, 52) for the flexible elongate medical element (15).

11. Envelope (30) according to claim 10, wherein the main portion (32A) comprises at least one transverse groove (42) extending perpendicularly to the longitudinal axis (X), hollowed out in the free surface having a concave shape, and
wherein at least one of the guide members (52) is configured to be positioned in the transverse groove (42) when the cover (50) is in the closed position.

12. Envelope (30) according to claim 10, wherein at least one of the complementary guide members (52) cooperates with the support member (44) so as to sandwich the flexible elongate medical element (15).

13. Envelope (30) according to claim 9, comprising electrically-controlled means for locking the cover (50) in the closed position.

14. Envelope (30) according to claim 13, wherein the means for locking the cover (50) in the closed position are connected to a presence sensor able to lock the cover (50) in the closed position when the presence sensor detects a presence close to the protective envelope (30).

15. Envelope (30) according to claim 1, comprising a pair of drive members (36) for the flexible elongate medical element (15), the drive members (36) being paired opposite one another, one on either side of the longitudinal axis (X) of the guide track (32).

16. Assembly comprising a robotic module (16) for driving a flexible elongate medical element (15) and a protective envelope (30) as claimed in claim 1, the envelope (30) being fixed to the robotic driving module (16) so as to form a barrier separating a space comprising the robotic module (16) from a space comprising the flexible elongate medical element (15).

17. Assembly according to claim 16, wherein the protective envelope (30) is removably attached to the robotic module (16).

18. Assembly according to claim 16, wherein the protective envelope (30) is sterile.

19. Assembly according to claim 16, wherein the robotic driving module (16) is configured to drive a catheter guide and/or a catheter through the envelope (30).

20. Envelope (30) according to claim 2, wherein the longitudinal groove (40) is part of a plane of symmetry (XZ) of the free surface having a concave shape.
